(19) United States
(12) Patent Application Publication
(10) Pub. No.: US 2010/0268031 A1
(43) Pub. Date: Oct. 21, 2010

(54) ELECTRIC BENDING ENDOSCOPE

(76) Inventor: Reiji KOYAMA, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/760,916

(22) Filed: Apr. 15, 2010

Related U.S. Application Data
(63) Continuation of application No. PCT/JP2009/067425, filed on Oct. 6, 2009.

(30) Foreign Application Priority Data
Oct. 22, 2008 (JP) 2008-272292

(51) Int. Cl. A61B 1/005

(52) U.S. Cl. 600/146

(57) ABSTRACT

An electrically bending endoscope includes a driven unit including an insertion portion extending in a longitudinally axial direction, and a driving unit to be attached to and detached from a proximal end portion of the driven unit. The driving unit includes a driving portion to produce driving force, and a driving transmission mechanism to transmit the driving force and including a driving coupling portion. The driven unit includes a bending portion provided in the insertion portion and to be operated to be bent by the driving force, and a driven transmission mechanism to transmit the driving force to the bending portion and including a driven coupling portion to be coupled with and separated from the driving coupling portion. The driving unit includes a detecting portion connected to the driving portion and to detect a load produced in the driving transmission mechanism corresponding to a load in the driven transmission mechanism.

### CROSS-REFERENCE TO RELATED APPLICATIONS

This is a Continuation Application of PCT Application No. PCT/JP2009/067425, filed Oct. 6, 2009, which was published under PCT Article 21(2) in Japanese.

This application is based upon and claims the benefit of priority from prior Japanese Patent Application No. 2008-272292, filed Oct. 22, 2008, the entire contents of which are incorporated herein by reference.

### BACKGROUND OF THE INVENTION

1. Field of the Invention

The present invention relates to an electrically bending endoscope wherein a bending portion is configured to be electrically operated to be bent.

2. Description of the Related Art

A manually bending endoscope is used as an endoscope wherein a bending portion is manually operated to be bent. For example, the endoscope includes an elongated insertion portion configured to be inserted into a cavity. A bending portion is provided in the distal end portion of the insertion portion and configured to be operated to be bent. In the bending portion, a large number of substantially circularly cylindrical bending parts are coaxially coupled with each other so as to be rotatable relative to each other. An operation portion is coupled to the proximal end portion of the insertion portion and configured to be held and operated by an operator. A bending operation knob is provided in the operation portion and configured to be operated to be rotated, and the bending operation knob is connected to a bending mechanism built-in the operation portion. An operation wire is wound around a sprocket of the bending mechanism, the one end side part and the other end side part of the operation wire are inserted through the operation portion and the insertion portion so as to be movable forward and backward, and the one end part and the other end part of the operation wire are fixed to the distal end bending part. When the bending operation knob is operated to be rotated, the sprocket is driven to be rotated, the one end side part and the other end side part of the operation wire are pulled and loosened, and the bending portion is operated to be bent. Here, depending on the condition of the bending portion such as an amount of bending of the bending portion and whether the bending portion is pushed by outside thing or not, resistance against the bending operation is changed and an amount of traction force applied to the operation wire is changed. An operator can estimate an amount of traction force applied to the operation wire on the basis of an amount of force of reaction torque against rotational or fixing operation to the bending operation knob, and recognize the condition of bending portion on the basis of the amount of traction force estimated.

One the other hand, as is disclosed in Jpn. Pat. Appln. KOKAI Publication Nos. 2000-279376 and 7-124104, an electrically bending endoscope is used as an endoscope wherein a bending portion is electrically operated to be bent. In the electrically bending endoscope, when a joystick, a trackball and the like are operated, an electric driving portion is operated which is built in the operation portion, and a sprocket is driven to be rotated by the electric driving portion. Here, in order to detect the bending condition of the bending portion, in the electrically bending endoscope disclosed in Jpn. Pat. Appln. KOKAI Publication No. 2000-279376, a tension sensor is provided in the distal end portion of the bending portion and configured to detect tension applied to the operation wire. Moreover, in the electrically bending endoscope disclosed in Jpn. Pat. Appln. KOKAI Publication No. 7-124104, a pressure-sensitive sensor is provided at the outer peripheral portion of the distal end portion of the bending portion.

### BRIEF SUMMARY OF THE INVENTION

In an aspect of the present invention, an electrically bending endoscope includes: a driven unit including an insertion portion extending in a longitudinally axial direction; and a driving unit configured to be attached to and detached from a proximal end portion of the driven unit. The driving unit includes: a driving portion configured to produce driving force; and a driving transmission mechanism configured to transmit the driving force produced by the driving portion and including a driving coupling portion. The driven unit includes: a bending portion provided in the insertion portion and configured to be operated to be bent by the driving force; and a driven transmission mechanism configured to transmit the driving force to the bending portion and including a driven coupling portion configured to be coupled with and separated from the driving coupling portion wherein the driven coupling portion is coupled with the driving coupling portion to transmit the driving force to the driven transmission mechanism from the driving transmission mechanism when the driven unit is attached to the driving unit. The driving unit includes a detecting portion connected to the driving portion of the driving unit and configured to detect a load produced in the driving transmission mechanism corresponding to a load in the driven transmission mechanism.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing an endoscope system according to a first embodiment of the present invention;

FIG. 2 is a perspective view showing an insertion portion unit according to the first embodiment of the present invention;

FIG. 3 is a schematic view showing an inner construction of the insertion portion unit according to the first embodiment of the present invention;

FIG. 4 is a perspective view showing a motor unit according to the first embodiment of the present invention;

FIG. 5 is a perspective view showing an inner construction of the motor unit according to the first embodiment of the present invention;

FIG. 6 is a perspective view showing a driving mechanism according to the first embodiment of the present invention;

FIG. 7 is a side view showing the driving mechanism according to the first embodiment of the present invention;

FIG. 8 is a transversally cross-sectional view showing the driving mechanism according to the first embodiment of the present invention;

FIG. 9 is a longitudinally cross-sectional view showing the driving mechanism according to the first embodiment of the present invention;

FIG. 10 is a perspective view showing a rack according to a second embodiment of the present invention;

FIG. 11 is a perspective view showing a clutch portion according to a third embodiment of the present invention;

FIG. 12 is a perspective view showing a rack and a rotationally axial portion according to a fourth embodiment of the present invention;

FIG. 13 is a cross-sectional view showing a clutch portion and a detecting portion according to a fifth embodiment of the present invention;

FIG. 14 is a cross-sectional view showing a clutch portion and a detecting portion according to a sixth embodiment of the present invention;

FIG. 15 is a cross-sectional view showing a clutch portion and a detecting portion according to a seventh embodiment of the present invention;

FIG. 16 is a cross-sectional view showing a clutch portion and a detecting portion according to an eighth embodiment of the present invention;

FIG. 17 is a cross-sectional view showing a clutch portion and a detecting portion according to a first modified example of the eighth embodiment of the present invention;

FIG. 18 is a cross-sectional view showing a clutch portion and a detecting portion according to a second modified example of the eighth embodiment of the present invention;

FIG. 19 is a longitudinally cross-sectional view showing a clutch portion and a detecting portion according to a ninth embodiment of the present invention;

FIG. 20 is a transversally cross-sectional view showing a clutch portion and a detecting portion according to the ninth embodiment of the present invention;

FIG. 21 is a cross-sectional view showing a transmission portion and a detecting portion according to a tenth embodiment of the present invention;

FIG. 22 is a perspective view showing a fixing gear member according to a modified example of the tenth embodiment of the present invention;

FIG. 23 is a perspective view showing a driving coupling according to an eleventh embodiment of the present invention;

FIG. 24 is a perspective view showing a driving coupling according to a twelfth embodiment of the present invention;

FIG. 25 is a perspective view showing a frame and a driving mechanism according to a thirteenth embodiment of the present invention;

FIG. 26 is a cross-sectional view showing a motor unit according to a fourteenth embodiment of the present invention;

FIG. 27 is a cross-sectional view showing an electrically bending endoscope according to a fifteenth embodiment of the present invention;

FIG. 28 is a perspective view showing an electrically bending endoscope according to a sixteenth embodiment of the present invention;

FIG. 29A is a cross-sectional view showing a driving assembly according to the sixteenth embodiment of the present invention along line XXIXA-XXIXA in FIG. 28;

FIG. 29B is a cross-sectional view showing a driving assembly according to a sixteenth embodiment of the present invention along line XXIXB-XXIXB in FIG. 28; and

FIG. 30 is a cross-sectional view showing a coupling and separating mechanism of a driving assembly and a supporting assembly according to a sixteenth embodiment of the present invention.

### DETAILED DESCRIPTION OF THE INVENTION

Hereinafter, each of embodiments of the present invention will be explained referring to the drawings.

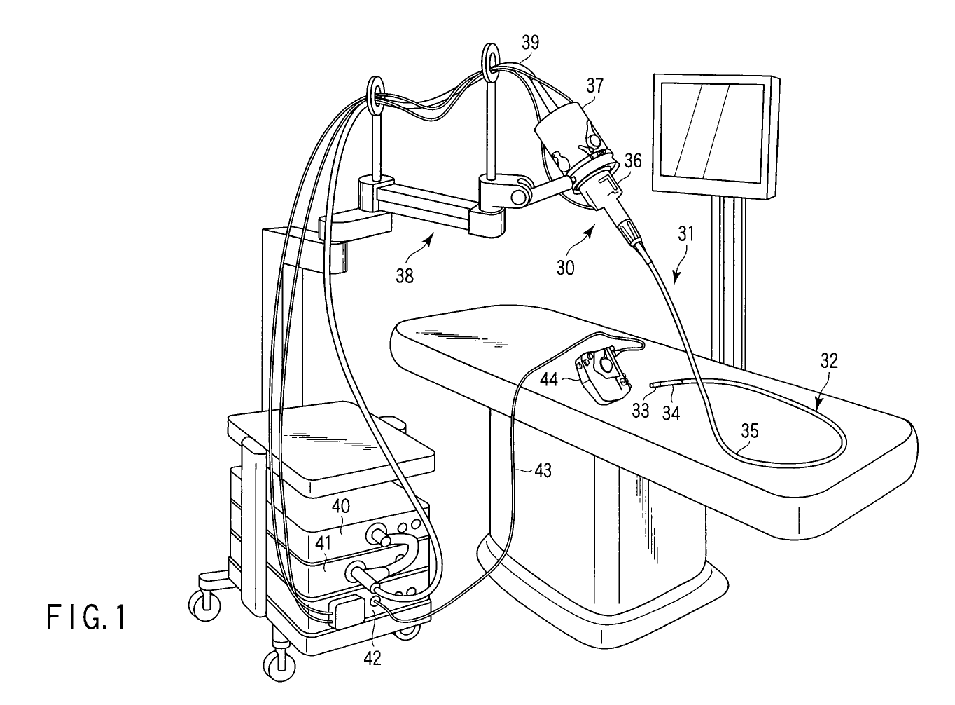

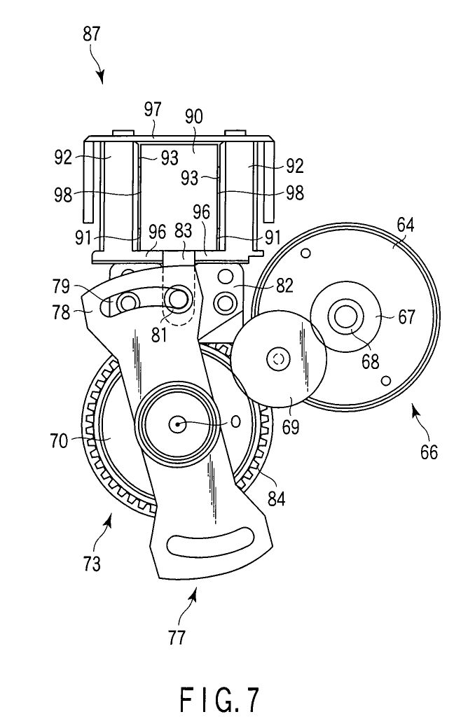

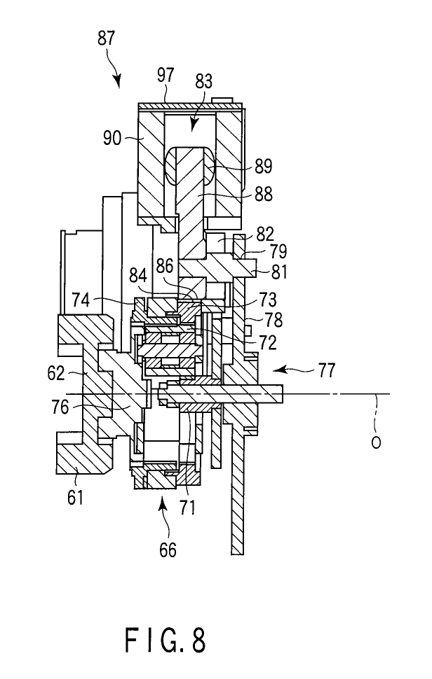

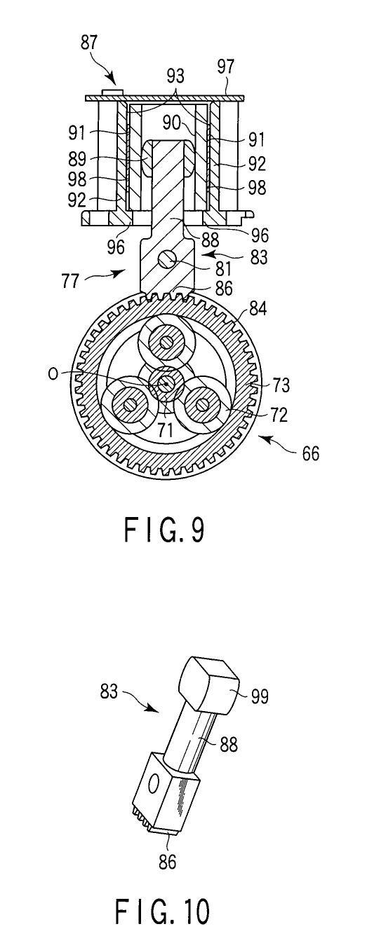

FIGS. 1 to 9 show a first embodiment of the present invention.

Referring to FIG. 1, an endoscope system will be explained.

The endoscope system includes a separate type of electrically bending endoscope 30. The electrically bending endoscope 30 includes an insertion portion unit 31 as a driven unit. The insertion portion unit 31 includes an elongated insertion portion 32 configured to be inserted into the cavity in the body. In the insertion portion 32, a distal end rigid portion 33, a bending portion 34 configured to be operated to be bent, and a flexible tube portion 35 being long and flexible are provided from the distal end side to the proximal end side. An attachment and detachment portion 36 is coupled to the proximal end portion of the insertion portion 32. The attachment and detachment portion 36 of the insertion portion unit 31 is configured to be attached to and detached from a motor unit 37 as a driving unit. The motor unit 37 is configured to produce driving force for operating the bending portion 34 to be bent. Moreover, the motor unit 37 is held by an endoscope holding apparatus 38 so as to be moved and fixed. A universal cable 39 extends from the motor unit 37, and the universal cable 39 is connected to a light source apparatus 40 and a video processor 41. The video processor 41 is connected to a system controller 42, and an operation unit 44 is connected to the system controller 42 via an operation cable 43. A bending operation switch and a change-over switch are provided in the operation unit 44, the bending operation switch is for operating the bending portion 34 to be bent, and the change-over switch is for changing over the bending portion 34 between an operable state and a releasing state.

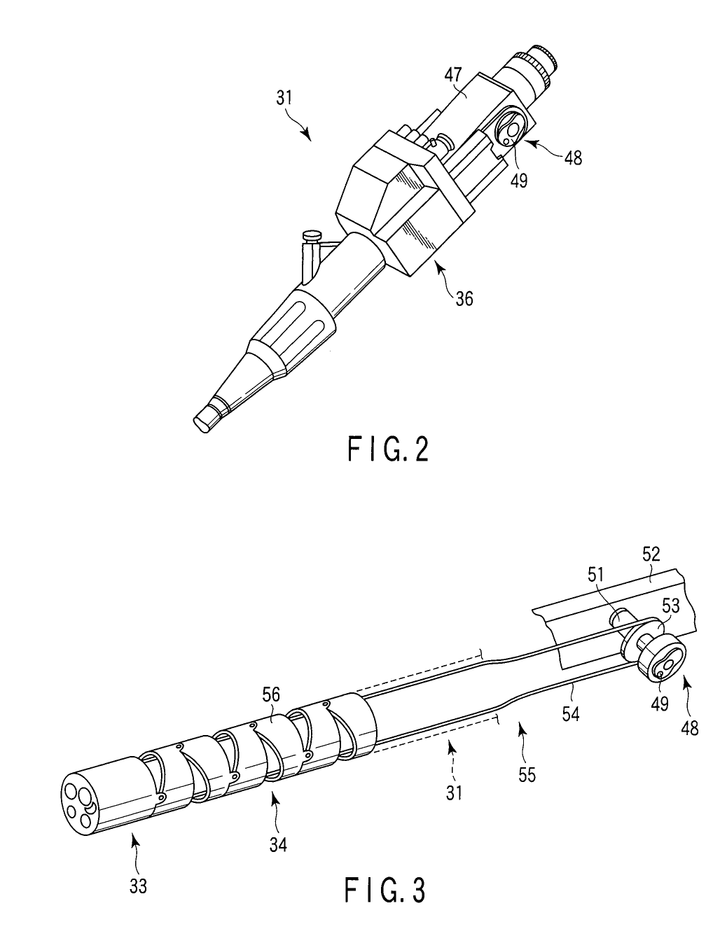

Referring to FIGS. 2 and 3, the insertion portion unit 31 will be explained in detail.

An insertion and extraction portion 47 is formed in the attachment and detachment portion 36 of the insertion portion unit 31 and configured to be inserted into and extracted from the motor unit 37. A driven coupling 48 as a driven coupling portion is provided in the insertion and extraction portion 47 and configured to be driven to be rotated by the motor unit 37. An engaging convex portion 49 extends in the terminal end portion of the driven coupling 48. A driven axial portion 51 is coaxially coupled to the root end portion of the driven coupling 48. The driven axial portion 51 is inserted into the insertion and extraction portion 47, and the inner end portion of the driven axial portion 51 is supported by a supporting wall 52 within the insertion and extraction portion 47 so as to be rotatable about the central axis of the driven axial portion 51. A sprocket 53 is fitted on the outside of and fixed to the middle portion of the driven axial portion 51. An operation wire 54 is wound around the sprocket 53. The one end side part and the other end side part of the operation wire 54 are inserted through the insertion portion unit 31 so as to be movable forward and backward and extend to the distal end portion of the bending portion 34, respectively. In the bending portion 34, a large number of substantially circular cylindrical bending parts 56 are coaxially coupled in order so as to be rotated relative to each other. The one end side part and the other end side part of the operation wire 54 are arranged symmetrically with each other relative to the central axis of the bending portion 34 in the bending portion 34, and the one end part and the other end part of the operation wire 54 are fixed to the distal end bending part 56, respectively. When the driven coupling 48 is driven to be rotated in the one direction and the other direction, the sprocket 53 is driven to be rotated in the one direction and the other direction, the one end side part and the other end side part of the operation wire 54 are pulled and loosened, or loosened and pulled, the bending portion 34 is operated to be bent in the one direction and the other direction opposite to each other, respectively. The pair of driven couplings 48, the sprockets 53 and the operation wires 54 having configurations similar to each other is used for bending operation in the up and down direction and the left and right direction. That is, the driven coupling 48, the sprocket 53 and the operation wire 54 form a driven transmission mechanism 55.

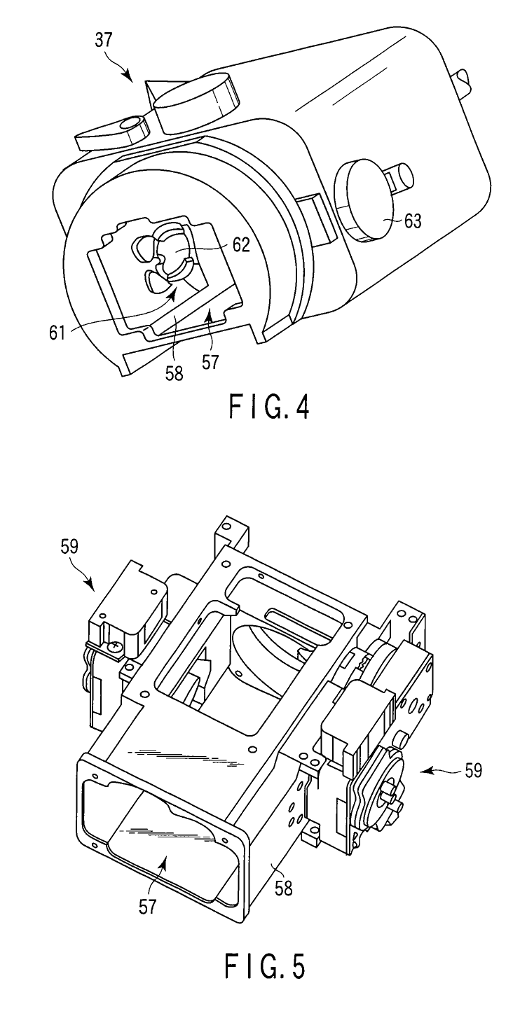

Referring to FIGS. 4 to 9, the motor unit 37 will be explained in detail.

Referring to FIGS. 4 and 5, an insertion and extraction bore 57 extends in the axial direction in the motor unit 37, and the insertion and extraction portion 47 of the insertion portion unit 31 is configured to be inserted into and extracted from the insertion and extraction bore 57. The insertion and extraction bore 57 is formed by the lumen of a substantially square-cylindrical frame 58. A driving mechanism 59 for bending-operation of the up and down direction and a driving mechanisms 59 for bending-operation of the left and right direction are provided at both the outer side portions of the frame 58, respectively. The driving coupling 61 as a driving coupling portion of the driving mechanism 59 is arranged within the insertion and extraction bore 57. An engaging concave portion 62 extends at the terminal end portion of the driving coupling 61. When the insertion and extraction portion 47 of the insertion portion unit 31 is inserted into and extracted from the insertion and extraction bore 57 of the motor unit 37, the engaging convex portion 49 of the driven coupling 48 is inserted into and extracted from the engaging concave portion 62 of the driving coupling 61, and the driven coupling 48 and the driving coupling 61 are coupled with and separated from each other. When the driving coupling 61 and the driven coupling 48 are coupled with each other, the driving force can be transmitted to the driven transmission mechanism 55 from the driving mechanism 59 via both the couplings. Here, a change-over lever 63 for clutch operation of the driving mechanism 59 is provided at the motor unit 37.

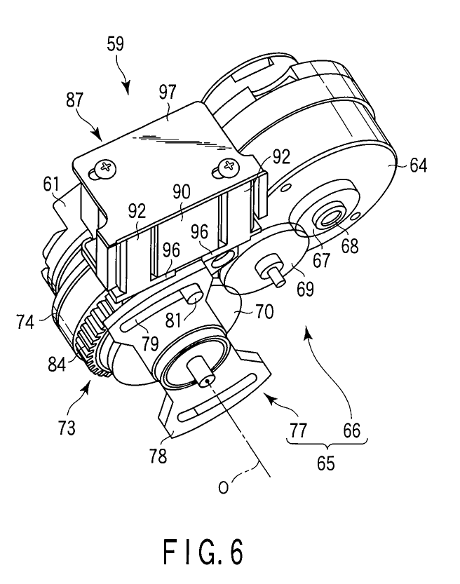

Referring to FIGS. 6 to 9, the driving mechanism 59 will be explained in detail.

The driving mechanism 59 includes a motor 64 as a driving portion configured to produce driving force, and a driving transmission mechanism 65 configured to transmit the driving force produced by the motor 64.

In the driving transmission mechanism 65, a transmission portion 66 is arranged between the motor 64 and the driving coupling 61 and formed by a gear train configured to transmit driving force. That is, a driving axial portion 68 of the motor 64 extends outwardly in the wide direction from the motor unit 37, and a driving gear 67 which is a spur gear is fixed to the terminal end portion of the driving axial portion 68. The driving gear 67 is engaged with an idler gear 69 which is a spur gear, and the idler gear 69 is engaged with a middle gear 70 which is a spur gear. The central axis of the middle gear 70 agrees with a rotational driving axis O. A sun gear 71 is coaxially fixed to the axially inside of the middle gear 70. A planetary gear 72 is engaged with the sun gear 71. A ring-shaped fixing gear 73 is arranged on the outside of the planetary gear 72, its central axis agreeing with the rotational driving axis O, and outer teeth of the planetary gear 72 are engaged with inner teeth which is that of a spur gear and formed in the fixing gear 73. The fixing gear 73 is configured to be changed over between a releasing state where the fixing gear 73 is rotatable about the rotational driving axis O and a fixing state where it is unrotatable. Moreover, a ring-shaped output gear 74 is arranged on the axially inside of the fixing gear 73, its central axis agreeing with the rotational driving axis O, and outer teeth of the planetary gear 72 are engaged with inner teeth like a spur gear formed in the output gear 74. The output gear 74 is rotatable about the rotational driving axis O. An output axial portion 76 is arranged on the inside of the output gear 74, its central axis agreeing with the rotational driving axis o, and the inner teeth of the output gear 74 are engaged with outer teeth like a spur gear formed in the output axial portion 76. A driving coupling 61 is fixed to the axially inner end portion of the output axial portion 76, its central axis agreeing with the rotational driving axis O. Then, when the fixing gear 73 is in a fixing state, driving force produced by the motor 64 can be transmitted to the driving coupling 61 via the gear train.

Moreover, a clutch portion 77 is formed in the driving transmission mechanism 65 and configured to change over the driving transmission mechanism 65 between a transmitting state where the driving transmission mechanism 65 can transmit driving force and a separation state where the driving transmission mechanism 65 cannot transmit driving force. That is, the clutch portion 77 includes a cam 78 configured to be rotated about the rotational driving axis O between a releasing position and a fixing position. The cam 78 is configured to be manually operated to be rotated by the change-over lever 63 of the motor unit 37 and configured to be electrically operated to be rotated by the change-over switch of the operation unit 44. A cam groove 79 extends in the peripheral direction of the rotational driving axis O of the cam 78, and a cam pin 81 as a fulcrum portion is inserted through the cam groove 79 parallel to the axial direction. A rack 83 as a fixing member is coupled to the axially inner end portion of the cam pin 81. The cam pin 81 is supported by a driving mechanism housing 82 so as to be unmovable in the peripheral direction and slidable in the radial direction, and rotatable about the central axis of the cam pin 81. When the cam 78 is arranged at the releasing position and the fixing position, the cam pin 81 and the rack 83 are arranged in a releasing position on the radially outside and a fixing position on the radially inside, respectively. A rack teeth portion 86 as a fixing member teeth portion is formed on the radially inner end portion of the rack 83. The rack 83 is arranged on the radially outside of the above fixing gear 73, and a fixing teeth portion 84 as a fixing gear teeth portion which is that of a spur gear is formed in the peripheral portion of the fixing gear 73. When the rack 83 is in the fixing position on the radially inside, the rack teeth portion 86 of the rack 83 is engaged with the fixing teeth portion 84 of the fixing gear 73 and the fixing gear 73 is in the fixing state where the fixing gear 73 is fixed. On the other hand, when the rack 83 is in the releasing position on the radially outside, the rack teeth portion 86 and the fixing teeth portion 84 are disengaged from each other, the fixing gear 73 is in the releasing state where the fixing gear 73 is rotatable, and the driving coupling 61 cannot transmit driving force produced by the motor 64 because the gear train idles.

Furthermore, the driving mechanism 59 includes a detecting portion 87 provided in the driving transmission mechanism 65 and configured to detect force applied to the driving transmission mechanism 65. That is, a rod portion 88 is formed on the radially outer side part of the rack 83 and has a shape of a quadrangular prism extending in the radial direction. A spherical member 89 as an application portion is fixed to the end portion of the rod portion 88. However, the radially inner end portion and the radially outer end portion of the spherical member 89 forms surfaces orthogonal to the radial direction, respectively. The radially outer side part of the rack 83 is inserted into a cylindrical block 90 extending in the radial direction. The inner peripheral surface of the block 90 has a circular transversely cross-section orthogonal to the radial direction and has the inner diameter a little larger than the outer diameter of the spherical member 89. The outer peripheral surface of the block 90 includes a pair of pushing surfaces 91 perpendicular to the tangential direction of the fixing gear 73. A pair of supporting walls 92 stands on the tangentially outside of the block 90 adjacent to the block 90. Supporting surfaces 93 are formed on the pair of supporting walls 92 adjacent to and facing the pushing surfaces 91 of the block 90, respectively. A board-shaped force sensor 98 is covered on each the supporting surface 93, and the force sensor 98 is held between the supporting surface 93 of the supporting wall 92 and the pushing surface 91 of the block 90. One of a load sell, a pressure sensor, a piezo element is used as the force sensor 98. Here, a board-shaped inner stopper 96 inwardly protrudes from the radially inner end portion of the supporting wall 92, and a board-shaped outer stopper 97 is covered on and is fixed to the radially outside of the pair of supporting walls 92. The inner stopper 96 and the outer stopper 97 are configured to limit radially movement of the block 90.

Next, detecting operation will be explained in the electrically bending endoscope 30 according to the present invention.

When the electrically bending endoscope 30 is used, the insertion and extraction portion 47 of the insertion portion unit 31 is inserted into the insertion and extraction bore 57 of the motor unit 37, and the insertion portion unit 31 is attached to the motor unit 37. Then, the change-over switch of the operation unit 44 is operated to make the bending portion 34 in the operable state. At this time, the cam 78 is rotated from the releasing position to the fixing position in the driving mechanism 59 of the motor unit 37, the cam pin 81 and the rack 83 is moved inwardly in the radial direction from the releasing position to the fixing position, the rack teeth portion 86 of the rack 83 is engaged with the fixing teeth portion 84 of the fixing gear 73, and the fixing gear 73 became in the fixing state. After that, the insertion portion 32 is inserted into the cavity in the body, and the bending operation switch of the operation unit 44 is operated to operate the bending portion 34 to be bent, if necessary. At this time, the driving force is produced by the motor 64 of the driving mechanism 59, and the driving force is transmitted to the driving coupling 61 via the gear train. Then, the driven coupling 48 is driven to be rotated by the driving coupling 61. When the driven coupling 48 is driven to be rotated, the sprocket 53 is driven to be rotated, the one end side part and the other end side part of the operation wire 54 is pulled and loosened, respectively, and the bending portion 34 is operated to be bent.

When the driven coupling 48 is driven to be rotated or fixed by the driving coupling 61, torque is applied as reaction to the driving coupling 61 and the output axial portion 76 from the driven coupling 48, and the torque is transmitted to the output gear 74. The torque applied to the output gear 74 corresponds to reaction torque sensed by an operator for estimating an amount of traction force when the bending operation knob is operated to be rotated or fixed in the manually bending endoscope. The torque transmitted to the output gear 74 is further transmitted to the planetary gear 72, and then, the fixing gear 73. The torque applied to the fixing gear 73 is detected as follows. That is, the rack teeth portion 86 on the radially inner end portion of the rack 83 is engaged with the fixing teeth portion 84 of the fixing gear 73, and force in the tangential direction of the fixing gear 73 is applied to the rack teeth portion 86 from the fixing gear 73. The rack 83 is rotatable about the central axis of the cam pin 81, the rack teeth portion 86 on the radially inner portion of the rack 83 functions a point of force, the cam pin 81 functions a fulcrum and the spherical member 89 on the radially outer end portion of the rack 83 functions a point of application, respectively, and the force is applied to the inner peripheral surface of the block 90 from the spherical member 89. Here, the distance between the point of force and the fulcrum is fixed and the distance between the fulcrum and the point of application is fixed, and therefore, a transmit rate of force is fixed and the force can be stably transmitted. Moreover, the force is transmitted from the spherical member 89 of the rack 83 to the inner peripheral surface of the block 90 with point contact, and therefore, the force can be transmitted toward the force sensor 98 without making both the members parallel to each other differing from the case where the force is transmitted with surface contact. The force applied to the inner peripheral surface of the block 90 is transmitted to the force sensor 98 from the pushing surface 91 to the outer peripheral surface of the block 90 via the block 90. That is, while force is transmitted to the block 90 from the spherical member 89 with the point contact, force can be transmitted to the sensor surface of the force sensor 98 from the pushing surface 91 of the block 90 with the surface contact. Here, the inner stopper 96 and the outer stopper 97 limits movement of the block 90 in the direction horizontal relative to the sensor surface of the force sensor 98, and therefore, shearing force horizontal relative to the sensor surface is not applied to the sensor surface of the force sensor 98 from the pushing surface 91 of the block 90. Moreover, even when the rack 83 is radially moved between the fixing position and the releasing position, the block 90 is prevent from being inclined by movement of the rack 83. According to a bending direction of the bending portion 34, the motor 64 is driven to be rotated in the one direction and the other direction, torque in the one direction and the other direction is applied to the fixing gear 73, respectively. The torque in the one direction and the other direction is detected by one and the other of both the force sensors 98, respectively. In this way, torque applied to the fixing gear 73 is detected by the force sensor 98.

In the electrically bending endoscope 30 according to the present embodiment, torque applied to the output gear 74 corresponds to reaction torque sensed by an operator when the bending operation knob is operated to be rotated or fixed in the manually bending endoscope, and the torque is transmitted to the planetary gear 72 from the output gear 74 via the fixing gear 73 and detected, and therefore, the detected torque is approximate to the reaction torque sensed by the operator in the manually bending endoscope. That is, the electrically bending endoscope 30 is realized which is capable of realizing a sense of operation similar to that of the manually bending endoscope.

Moreover, in the electrically bending endoscope 30, the insertion portion unit 31 is shorter in the life than the motor unit 37, and insertion portion units 31 are used for one motor unit 37. The detecting portion 87 is provided in the motor unit 37 and not the insertion portion unit 31, and therefore, it is possible to reduce manufacturing costs for the whole system.

FIG. 10 shows a second embodiment of the present invention.

In a rack 83 according to the present embodiment, in stead of the spherical member 89, a short round column member 99 as an application portion is used which extends in the axial direction of a fixing gear 73. The radially inner end portion and the radially outer end portion of the short round column member 99 form the surfaces orthogonal to the radial direction, respectively. The inner peripheral surface of a block 90 has a square cross-section orthogonal to the radial direction corresponding to the shape of the short round column member 99. Even the short round column member 99 can transmit force in a similar manner to that by the spherical member 89.

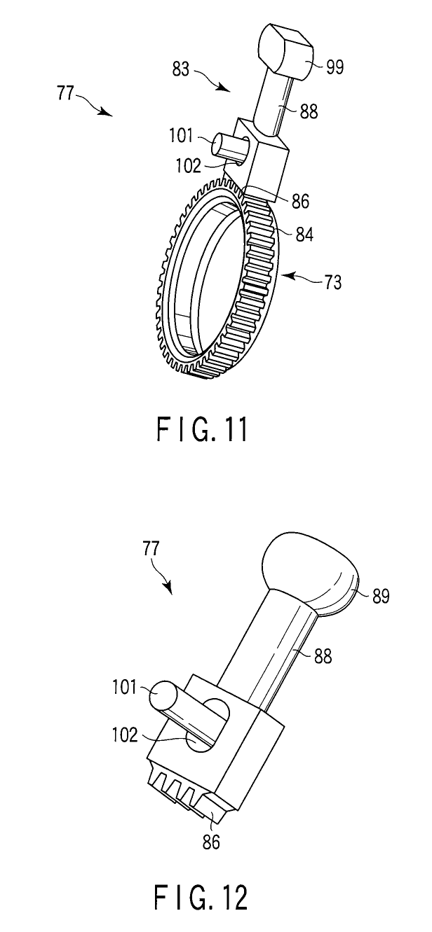

FIG. 11 shows a third embodiment of the present invention.

Differing from the clutch portion 77 according to the first embodiment, the cam 78 and the cam pin 81 are not used in a clutch portion 77 according to the present embodiment. A penetrating bore 102 is formed in a rack 83 in the axial direction of a fixing gear 73, and a rotationally axial portion 101 as a fulcrum portion is inserted through the penetrating bore 102 of the rack 83. A rotationally axial portion 101 extends parallel to the central axis of the fixing gear 73 and fixed to a driving mechanism housing 82. The rack 83 is configured to slide in the axial direction of the fixing gear 73 along the rotationally axial portion 101 and to be arranged in a fixing position where the rack 83 is aligned with the fixing gear 73 and a rack teeth portion 86 and a fixing teeth portion 84 are engaged with each other and in a releasing position where the rack 83 is not aligned with the fixing gear 73 and the rack teeth portion 86 and the fixing teeth portion 84 is disengage from each other, by a manual or electrical moving mechanism. Here, an opening portion is formed on the axially one side of the fixing gear 73 in the block 90 in order not to prevent the axially movement of the rack 83. The rack 83 is rotatable about the rotationally axial portion 101 in the fixing position and torque applied to the fixing gear 73 can be detected via the rack 83 as is similar to the first embodiment.

FIG. 12 shows a fourth embodiment of the present invention.

Deferring from the clutch portion 77 according to the first embodiment, the cam 78 and the cam pin 81 are not used in a clutch portion 77 according to the present embodiment. A penetrating bore 102 is formed in a rack 83 in the axial direction of a fixing gear 73, and the penetrating bore 102 extends in the radial direction of the fixing gear 73. A rotationally axial portion 101 is inserted through a penetrating bore 102 of the rack 83. The rotationally axial portion 101 extends parallel to the central axis of the fixing gear 73 and fixed to a driving mechanism housing 82. The rack 83 is configured to slide in the radial direction relative to the rotationally axial portion 101 by a manual or electrical moving mechanism and to be arranged in a fixing position and a releasing position as is similar to the first embodiment. The rack 83 is rotatable about the rotationally axial portion 101 in the fixing position, and torque applied to the fixing gear 73 can be detected via the rack 83 as is similar to the first embodiment.

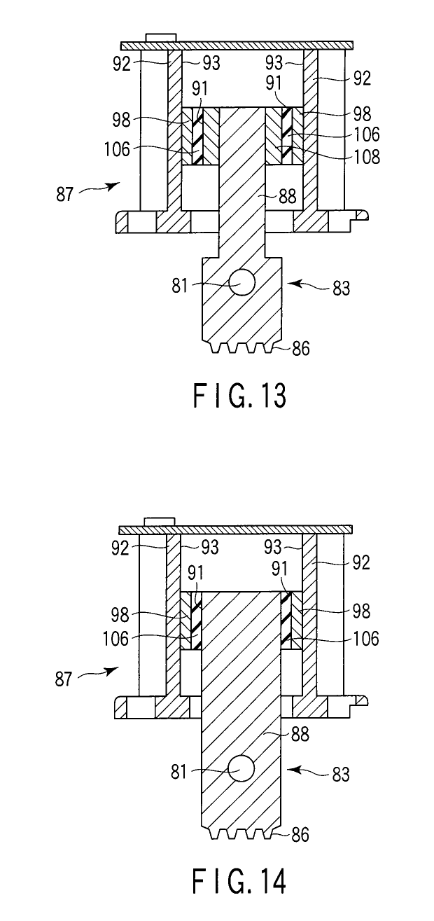

FIG. 13 shows a fifth embodiment of the present invention.

In a detecting portion 87 according to the present embodiment, a rod portion 88 of a rack 83 is slidably inserted into a rectangularly cylindrical supporting member 108 extending in the radial direction of a fixing gear 73. The outer peripheral surface of the supporting member 108 includes a pair of pushing surfaces 91 orthogonal to the tangential direction of the fixing gear 73. A board-shaped elastic member 106 is compressed and provided between the pushing surface 91 of the supporting member 108 and a force sensor 98 covered on a supporting surface 93 of a supporting wall 92. Preload is applied to the force sensor 98 by the elastic member 106. Therefore, dead zone of the force sensor 98 can be eliminated.

On the other hand, a force sensor 98 may be covered on a pushing surface 91 of a supporting member 108, and an elastic member 106 may be compressed and provided between a force sensor 98 and a supporting surface 93 of a supporting wall 92.

FIG. 14 shows a sixth embodiment of the present invention.

Deferring from the detecting portion 87 according to the fifth embodiment, the supporting member 108 is not used in a detecting portion 87 according to the present embodiment, and an elastic member 106 is compressed and provided between a rod portion 88 of a rack 83 and a force sensor 98. The rod portion 88 is slidable relative to the elastic member 106.

On the other hand, a force sensor 98 may be covered on a rod portion 88 of a rack 83, and an elastic member 106 may be compressed and provided between the force sensor 98 and a supporting surface 93 of a sensor cover 104. In this case, the rod portion 88 of the rack 83 may be slidable relative to the force sensor 98.

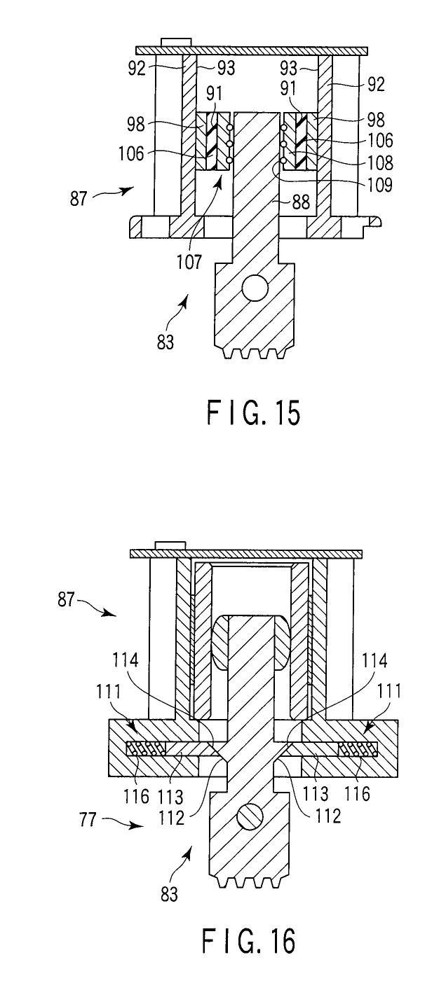

FIG. 15 shows a seventh embodiment of the present invention.

In a detecting portion 87 according to the present embodiment, as is similar to the detecting portion 87 according to the fifth embodiment, a rod portion 88 of a rack 83 is inserted into a supporting member 108. Rollers 109 are provided at the inner peripheral portion of the supporting member 108 in order to make it easy that sliding of the rod portion 88. The supporting member 108 and the rollers 109 form a slide unit 107. In change-over operation of a clutch portion 77, the rack 83 is moved in the radial direction and the rod portion 88 of the rack 83 slides utilizing the roller 109 in the supporting member 108, and therefore, the rack 83 can be decreased in the slide resistance.

Here, in stead of the slide unit 107, one of a bearing, a slide guide and so on may be used.

FIG. 16 shows an eighth embodiment of the present invention.

In a clutch portion 77 according to the present embodiment, a rack 83 is configured to be urged outwardly in the radial direction by an urging mechanism 111 in a clutch portion 77 similar to the first embodiment. That is, a taper receiving surface 112 is formed at a rod portion 88 of the rack 83 and spread outwardly in the radial direction. A taper surface 114 is formed at a push-out member 113, spread outwardly in the radial direction, and is slidably in contact with the taper receiving surface 112. The push-out member 113 is configured to be urged toward the rod portion 88 of the rack 83 in the tangential direction of the fixing gear 73 by an elastic urging member 116. In separation operation of the clutch portion 77, the rack 83 is moved outwardly in the radial direction by a cam pin 81, and the rack 83 is urged outwardly in the radial direction by the urging mechanism 111, and therefore, the rack 83 can be moved stably and smoothly.

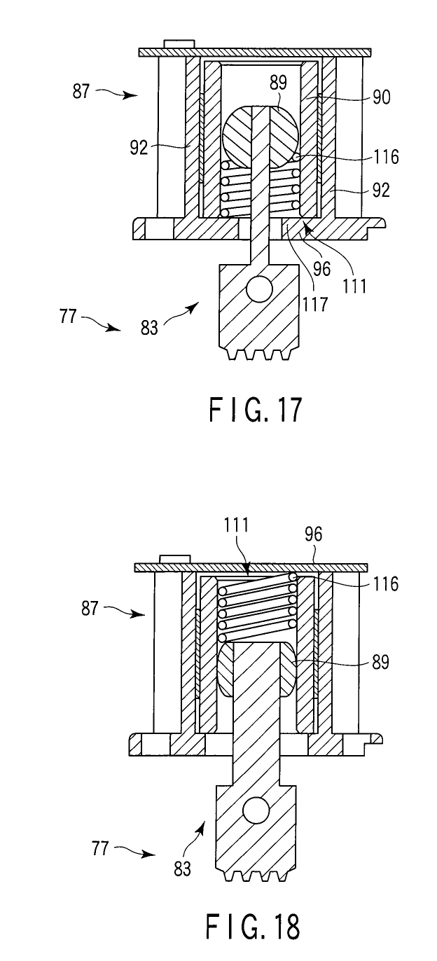

FIG. 17 shows a first modified example of the eighth embodiment of the present invention.

In an urging mechanism 111 according to the present modified example, an inner stopper 96 of a supporting wall 92 protrudes inwardly more than the inner peripheral surface of a block 90, the protruding end portion of the inner stopper 96 forms a spring receiver 117. An urging member 116 is compressed and provided between a spring receiver 117 and a spherical member 89 of a rack 83, and the urging member 116 is configured to urge the rack 83 outwardly in the radial direction.

FIG. 18 shows a second modified example of the eighth embodiment of the present invention.

In an urging mechanism 111 according to the present modified example, an urging member 116 is tensioned and provided between an outer stopper 97 and a spherical member 89 of a rack 83, and the urging member 116 is configured to urge the rack 83 outwardly in the radial direction.

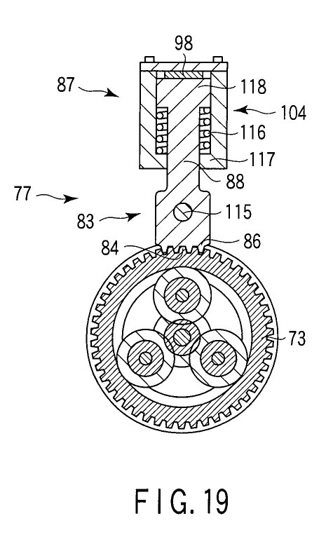

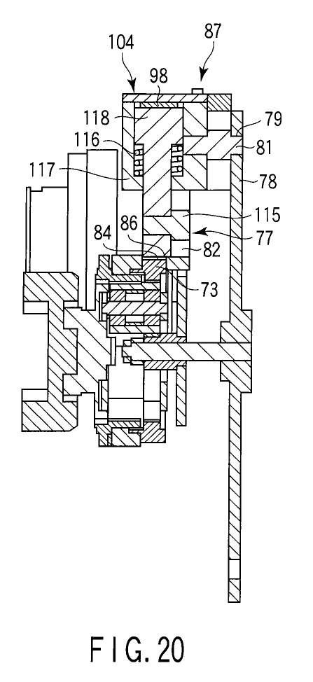

FIGS. 19 and 20 show a ninth embodiment of the present invention.

Differing from the clutch portion 77 and the detecting portion 87 according to the first embodiment, in stead of the cam pin 81, a sliding axial portion 115 is coupled to a rack 83 in a clutch portion 77 and a detecting portion 87 according to the present embodiment. The sliding axial portion 115 is supported by a driving mechanism housing 82 so as to be slidable in the radial direction of a fixing gear 73, and the rack 83 is slidable in the radial direction together with the sliding axial portion 115. A rod portion 88 of the rack 83 is inserted into a sensor cover 104 having a shape of a rectangular cylinder whose radially outer end portion is closed and slidable in the radial direction. A large-diameter portion 118 is formed at the distal end portion of the rod portion 88 of the rack 83, and a spring receiver 117 is formed at the radially inner portion of the sensor cover 104 and protrudes inwardly, and an elastic member 106 is compressed and provided between the large-diameter portion 118 of the rack 83 and the spring receiver 117 of the sensor cover 104. That is, the rack 83 is configured to be urged outwardly in the radial direction relative to the sensor cover 104. A force sensor 98 is covered on the inner surface of the radially outer end wall of the sensor cover 104, and the large-diameter portion 118 of the rack 83 is in contact with the force sensor 98. The cam pin 81 is coupled to the sensor cover 104, and the sensor cover 104 is radially slidable between a fixing position on the radially inside and a releasing position on the radially outside together with the cam pin 81. When the sensor cover 104 is arranged in the fixing position and the releasing position, the rack 83 is arranged in the fixing position and the releasing position via the elastic member 106, respectively. The rack 83 and the sensor cover 104 are unmovable in the tangential direction of the fixing gear 73. Therefore, when the rack 83 is in the fixing position and the fixing gear 73 is rotated, a fixing teeth portion 84 of the fixing gear 73 and a rack teeth portion 86 of the rack 83 are interacted with each other, force outwardly in the radial direction is applied to the rack 83, and the force outwardly in the radial direction is applied to the force sensor 98 via the rack 83. Torque applied to the fixing gear 73 is detected on the basis of the force applied to the force sensor 98.

In the present embodiment, it is possible for the one force sensor 98 to detect torque applied to the fixing gear 73 in both directions. Moreover, the force sensor 98 and the rack 83 are united, and therefore, it is easily feasible to exchange, in particular, the force sensor 98, and then, maintain the motor unit 37.

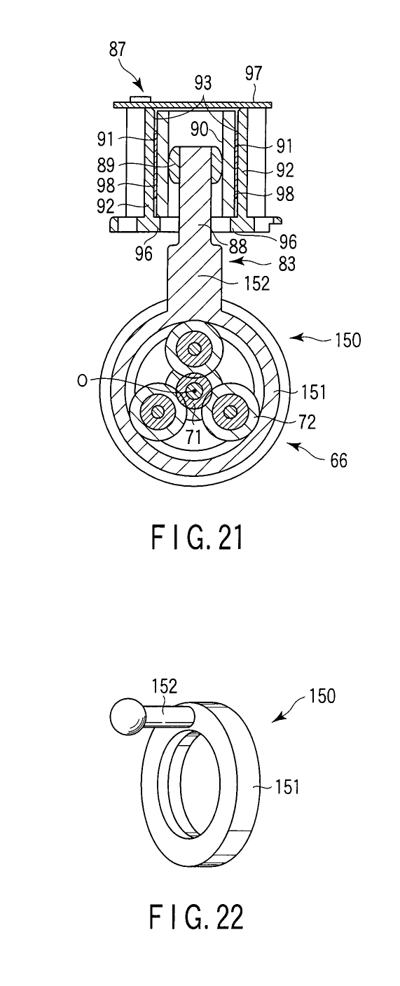

FIG. 21 shows a tenth embodiment of the present invention.

An electrically bending endoscope 30 according to the present embodiment does not have a clutch function, and a driving transmission mechanism 65 includes a transmission portion 66 and a detecting portion 87 similar to the first embodiment and does not include a clutch portion 77. That is, in the transmission portion 66 according to the present embodiment, a rack-integrated fixing gear 150 is used. In the rack-integrated fixing gear 150, a rack portion 152 as a rack protrudes outwardly in the radial direction integrally from the peripheral portion of a fixing gear portion 151 as a fixing gear. The fixing gear portion 151 and the rack portion 152 have configurations substantially similar to those of the fixing gear 73 and the rack 83 according to the first embodiment, but the fixing teeth portion 84 and the rack teeth portion 86 are not formed in the fixing gear portion 151 and the rack portion 152, respectively. Moreover, the cam pin 81 is not inserted through the rack portion 152. A spherical member 89 of the rack portion 152 is always supported by a pair of supporting walls 92 via a block 90 and a force sensor 98 with respect to the peripheral direction of the fixing gear portion 151, and the rack-integrated fixing gear 150 is always kept in a fixing state. As is similar to the first embodiment, torque transmitted to the fixing gear portion 151 is transmitted to the rack portion 152 from the fixing gear portion 151, and then transmitted to the force sensor 98 via the block 90 from the spherical member 89 of the rack portion 152. As is mentioned above, the torque applied to the fixing gear portion 151 is detected by the force sensor 98.

FIG. 22 shows a first modified example of a tenth embodiment of the present invention.

In a rack-integrated fixing gear 150 according to the present modified example, a rack portion 152 protrudes in the axial direction of the fixing gear portion 151 integrally from one ring-shaped end surface of a fixing gear portion 151. A detecting portion 87 similar to the tenth embodiment is formed for the rack portion 152, and torque applied to the fixing gear portion 151 is detected by a force sensor 98.

Various detecting portions may be used as a detecting portion configured to detect torque applied to a fixing gear 73 as well as the above mentioned embodiment. For example, the peripheral portion of a fixing gear 73 may be formed as a sprocket, a chain may be wound around the fixing gear 73 and a sprocket for detection, and torque applied to the fixing gear 73 may be detected on the basis of torque and the like of the sprocket for detection necessary for keeping the fixing gear 73 in a fixing state. Moreover, a gear for detection provided at a driving axis of a motor for detection may be engaged with a fixing teeth portion 84 of a fixing gear 73, and torque applied to the fixing gear 73 may be detected on the basis of current value and the like of the motor for detection necessary for keeping the fixing gear 73 in a fixing state. Furthermore, a torque meter is directly provided at a fixing gear 73. Moreover, torque applied to an output axial portion 76 may be detected by a rotational torque sensor of non-contact type.

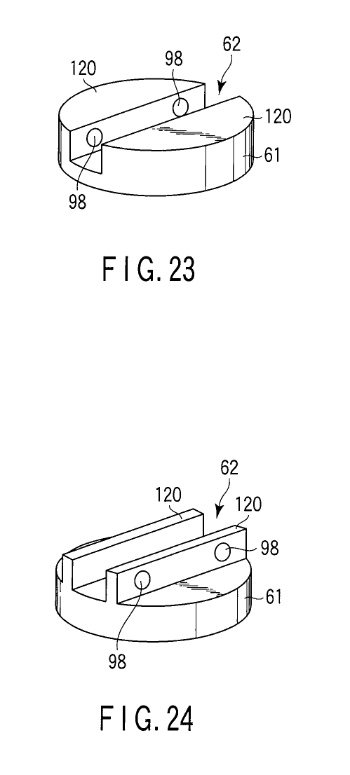

FIG. 23 shows an eleventh embodiment of the present invention.

In a driving coupling 61 according to the present embodiment, an engaging concave portion 62 is formed by a pair of wall portions 120. Force sensors 98 are covered on both the end portions of the inside surfaces of the pair of wall portions 120, respectively. The wall portions 120 are thick and have high rigidity, and a load sell and the like is used as a force sensor 98, which directly detect force. When a driven coupling 48 is driven to be rotated by the driving coupling 61, torque is applied as a reaction to a driving coupling 61 from the driven coupling 48, the torque is detected by the force sensors 98. The torque detected by the force sensors 98 is approximate to reaction torque sensed by an operator in a manually bending endoscope.

FIG. 24 shows a twelfth embodiment of the present invention.

Differing from the eleventh embodiment, force sensors 98 are covered on both the end portions of the outside surfaces of a pair of wall portions 120, respectively. The wall portions 120 are thin and have a low rigidity, and a strain gage configured to detect force by detecting flexure is used as the force sensor 98.

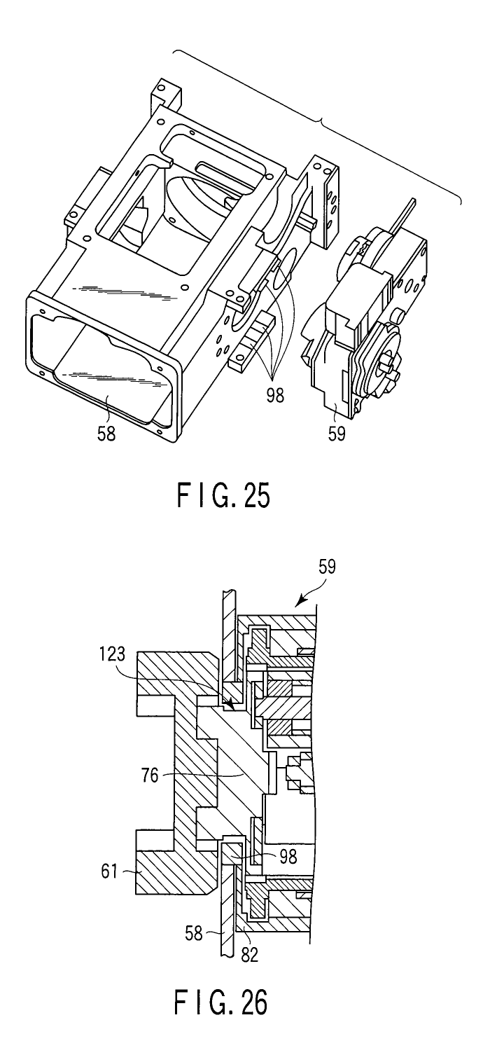

FIG. 25 shows a thirteenth embodiment of the present invention.

In an electrically bending endoscope 30 according to the present embodiment, a force sensor 98 is provided between a frame 58 as a supporting portion of a motor unit 37 and a driving mechanism 59. When a driven coupling 48 is driven to be rotated or fixed by the driving mechanism 59, torque is applied as reaction force to the driving mechanism 59 from the driven coupling 48. The torque is detected by the force sensor 98 between the driving mechanism 59 and the frame 58. The torque detected by the force sensor 98 is approximate to reaction torque sensed by an operator in a manually bending endoscope.

FIG. 26 shows a fourteenth embodiment of the present invention.

In an electrically bending endoscope 30 according to the present embodiment, an insertion bore 123 is formed in a motor unit 37, and an output axial portion 76 of a driving mechanism 59 is inserted through the insertion bore 123. The one end side part of a cylindrical force sensor 98 is inserted into and fixed to the inner peripheral surface of a frame 58 limiting the insertion bore 123. The other end portion of the force sensor 98 is fixed to a driving mechanism housing 82 of the driving mechanism 59. The output axial portion 76 is inserted through the force sensor 98, and a torque sensor is used as the force sensor 98. When a driven coupling 48 is driven to be rotated or fixed by the driving mechanism 59, reaction torque is applied to the driving mechanism 59 from the driven coupling 48, and the torque is detected by the force sensor 98 between the driving mechanism 59 and the frame 58.

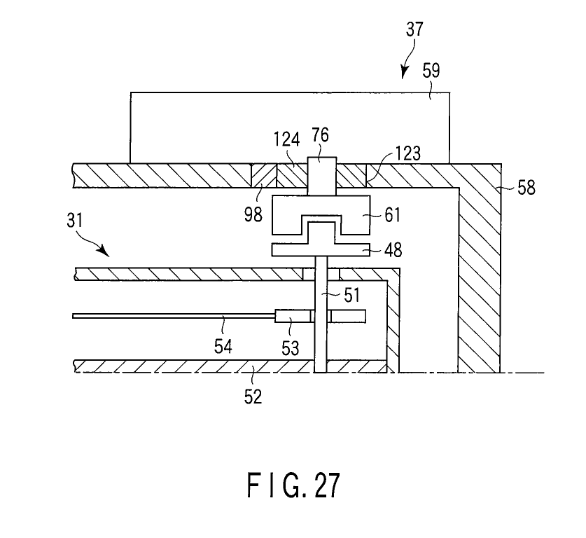

FIG. 27 shows a fifteenth embodiment of the present invention.

In an electrically bending endoscope 30 according to the present embodiment, as is similar to the fourteenth embodiment, an insertion bore 123 is formed in a motor unit 37, and an output axial portion 76 of a driving mechanism 59 is inserted through the insertion bore 123. A bearing 124 is fitted on the outside of the output axial portion 76, and the bearing 124 is arranged within the insertion bore 123. A force sensor 98 is provided between the bearing 124 and a frame 58. The force sensor 98 is arranged on the axially distal end side of the motor unit 37 relative to the bearing 124. When a driven coupling 48 is driven to be rotated by the driving mechanism 59, a sprocket 53 is driven to be rotated via a driven axial portion 51, and an operation wire 54 is pulled toward the proximal end side. Due to reaction to the sprocket 53 from the operation wire 54, the sprocket 53 is pulled toward the distal end side, the middle portion of the driven axial portion 51 to which the sprocket 53 is fixed is pulled toward the distal end side relative to the inner end portion thereof supported by a supporting wall 52, the driven coupling 48 of the terminal end portion of the driven axial portion 51 is urged toward the distal end side, and the driving mechanism 59 is urged toward the distal end side by the driven coupling 48. The urging force applied to the driving mechanism 59 is detected by the force sensor 98 provided between the bearing 124 and the frame 58. The urging force detected by the force sensor 98 corresponds to an amount of traction force of the operation wire 54.

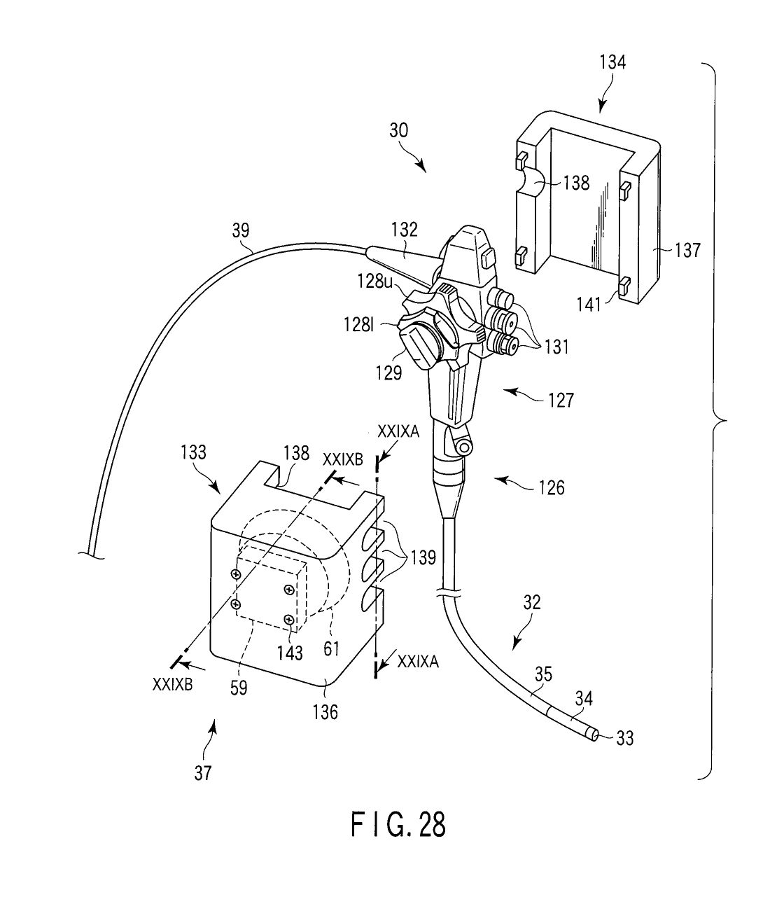

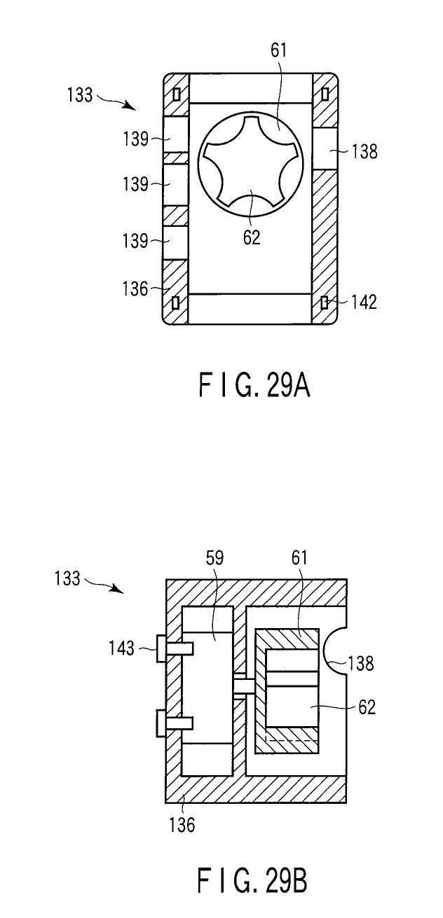

FIGS. 28 to 30 show a sixteenth embodiment of the present invention.

Referring to FIG. 28, an electrically bending endoscope 30 according to the present embodiment includes a manually bending endoscope 126 as a driven unit. The manually bending endoscope 126 includes an insertion portion 32 similar to the insertion portion 32 of the electrically bending endoscope 30 according to the first embodiment. An operation portion 127 is coupled to the proximal end portion of the insertion portion 32 and configured to be held and operated by an operator. In the operation portion 127, a UD and a LR bending operation knob 128u and 128l is provided on the side surface side in the wide direction and configured to operate a bending portion 34 to be bent in the up and down and the left and right direction, respectively. The UD and the LR bending operation knob 128u and 128l is arranged side by side coaxially with each other for a rotational axis extending in the wide direction such that the UD bending operation knob 128u is on the root end side and the LR bending operation knob 128l is on the terminal end side, and they are rotatable about the rotational axis and configured to be manually operated to be rotated and fixed. A fixing lever 129 is provided on the terminal end side of the LR bending operation knob 128l and configured to keep both the bending operation knobs 128u and 128l in a fixing state. When viewing in the axial direction, an outer shape of the LR bending operation knob 128l and the fixing lever 129 is included within an outer shape of the UD bending operation knob 128u. A driven transmission mechanism similar to that of the first embodiment is provided between the bending operation knobs 128u and 128l and the bending portion 34, and the bending portion 34 is operated to be bent by operating the bending operation knob 128u, 128l to be rotated. Moreover, various switches 131 for operating an endoscope system protrudes from the front side in the forward and backward direction of the operation portion 127, and an universal cable 39 extends from the back side in the forward and backward direction. Here, a break-preventing portion 132 is provided on the outside of the root end portion of the universal cable 39 and configured to prevent the universal cable 39 from being broken relative to the operation portion 127.

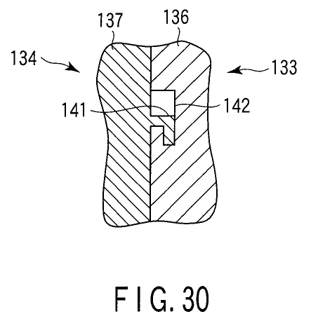

Referring to FIGS. 28 to 30, a motor unit 37 is configured to be attached to and detached from the operation portion 127 of the manually bending endoscope 126. The motor unit 37 is formed by a driving assembly 133 and a supporting assembly 134. The driving assembly 133 and the supporting assembly 134 are configured to be attached to and detached from the one side surface side and the other side surface side of the operation portion 127, respectively, and the driving assembly 133 and the supporting assembly 134 are configured to be coupled to and separated from each other when they are attached to and detached from the operation portion 127. That is, a driving assembly frame 136 of the driving assembly 133 and a supporting assembly frame 137 of the supporting assembly 134 have substantially U-shaped transverse cross-sections, respectively. When the driving assembly 133 and the supporting assembly 134 are attached to the operation portion 127 and coupled with each other, the driving assembly frame 136 and the supporting assembly frame 137 form a rectangular cylinder extending in the longitudinal direction of the operation portion 127 wherein the front and the back walls thereof are in contact with each other. Notch portions 138 for the break-preventing portion are formed at the end surface portions of the back walls of the driving assembly frame 136 and the supporting assembly frame 137, respectively, and notch portions 139 for the switches are formed at the end surface portion of the front wall of the driving assembly frame 136. In the attaching and coupling state of the driving assembly 133 and the supporting assembly 134, the break-preventing portion 132 is housed and held in both the notch portions 138 for the break-preventing portion and each switch 131 is housed in each notch portion 139 for the switch. Furthermore, hooks 141 are provided at the end surface portions of the front and the back wall of the supporting assembly frame 137, and hook holes 142 are formed at the end surface portions of the front and the back wall of the driving assembly frame 136. In the attaching and coupling state of the driving assembly 133 and the supporting assembly 134, as is shown in FIG. 30, the hooks 141 of the supporting assembly frame 137 are engaged with the hook holes 142 of the driving assembly frame 136, and it is prevented that the driving assembly 133 and the supporting assembly 134 are separated from each other and fallen off from the operation portion 127.

In the driving assembly 133, a driving mechanism 59 similar to the driving mechanism 59 according to the first embodiment is fixed to the inner surface of the side wall of the driving assembly frame 136 by screws 143 and the like. An engaging concave portion 62 is formed at the terminal end surface of a driving coupling 61 and configured to be engaged with the UD bending operation knob 128u as a driven coupling portion. That is, the inner shape of the engaging concave portion 62 is substantially the same shape of the outer shape of the UD bending operation knob 128u in the transverse cross-section orthogonal to the axial direction. In the attaching and coupling state of the driving assembly 133 and the supporting assembly 134, the driving coupling 61 is arranged coaxially with the UD bending operation knob 128u, the UD bending operation knob 128u is fitted on the engaging concave portion 62 of the driving coupling 61, and the driving coupling 61 and the UD bending operation knob 128u are coupled with each other. In this state, the UD bending operation knob 128u can be driven to be rotated by the driving coupling 61. Here, the fixing lever 129 and the LR bending operation knob 128l are housed within the engaging concave portion 62 without interfere with the driving coupling 61.

When using the electrically bending endoscope 30, the motor unit 37 is attached to the operation portion 127 of the manually bending endoscope 126, the UD bending operation knob 128u is fitted on the engaging concave portion 62 of the driving coupling 61, and the UD bending operation knob 128u and the driving coupling 61 are coupled with each other. When the UD bending operation knob 128u is driven to be rotated or fixed by the driving coupling 61, torque is applied as reaction to the driving coupling 61 from the UD bending operation knob 128u. The torque is reaction torque itself sensed by an operator when the UD bending operation knob 128u is operated to be rotated or fixed in the manually bending endoscope 126. As is similar to the first embodiment, torque approximate to the reaction torque is detected by a detecting portion 87 of the driving mechanism 59.

In the electrically bending endoscope 30 according to the present embodiment, it is possible to detect torque approximate to reaction torque sensed by an operator when the bending operation knob 128u is operated to be rotated or fixed in the manually bending endoscope 126. Moreover, the detecting portion 87 is provided in the driving unit 37 and not the manually bending endoscope 126, and therefore, the electrically bending endoscope 30 can be constructed by using the conventional manually bending endoscope 126 as it is.

Here, in the present embodiment, a driving mechanism similar to that of the second to the twelfth embodiment may be used, and a force sensor may be arranged between the driving mechanism and the driving assembly frame as a supporting portion, as is similar to the thirteenth to the fifteenth embodiment. Moreover, a motor unit may be used which includes no supporting assembly and is formed by only the driving assembly.

In each of the above-mentioned embodiments, a load sell, a pressure sensor, a piezo element and the like configured to directly detect force, a strain gage and a liner scale configured to indirectly detect force, and so on may be used as a force sensor.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An electrically bending endoscope comprising:
a driven unit including an insertion portion extending in a longitudinally axial direction; and
a driving unit configured to be attached to and detached from a proximal end portion of the driven unit, and
wherein the driving unit includes:
a driving portion configured to produce driving force; and
a driving transmission mechanism configured to transmit the driving force produced by the driving portion and including a driving coupling portion,
the driven unit includes:
a bending portion provided in the insertion portion and configured to be operated to be bent by the driving force; and
a driven transmission mechanism configured to transmit the driving force to the bending portion and including a driven coupling portion configured to be coupled with and separated from the driving coupling portion wherein the driven coupling portion is coupled with the driving coupling portion to transmit the driving force to the driven transmission mechanism from the driving transmission mechanism when the driven unit is attached to the driving unit, and
the driving unit includes a detecting portion connected to the driving portion of the driving unit and configured to detect a load produced in the driving transmission mechanism corresponding to a load in the driven transmission mechanism.

2. The electrically bending endoscope according to claim 1,
wherein the driven unit includes an attachment and detachment portion provided at the proximal end portion of the insertion portion and configured to be attached to and detached from the driving unit, and
a separate type of electrically bending endoscope is formed by the driving unit and the driven unit.

3. The electrically bending endoscope according to claim 1,
wherein the driven unit is a manually bending endoscope and includes: an operation portion provided at the proximal end portion of the insertion portion; and a bending operation knob provided at the operation portion and configured to operate the bending portion to be bent, and
the driving unit is configured to be attached to and detached from the operation portion and the driven coupling portion is formed by the bending operation knob.

4. The electrically bending endoscope according to claim 1,
wherein the detecting portion is provided in the driving transmission mechanism and configured to detect force applied to the driving transmission mechanism.

5. The electrically bending endoscope according to claim 4,
wherein the driving transmission mechanism includes a gear train configured to transmit the driving force,
the gear train includes a fixing gear configured to be kept in a fixing state where the fixing gear is unrotatable about a central axis of the fixing gear at least during transmission of the driving force, and
the detecting portion is configured to detect torque applied to the fixing gear when the fixing gear is in the fixing state.

6. The electrically bending endoscope according to claim 5,
wherein the driving transmission mechanism includes a fixing member configured to keep the fixing gear in the fixing state, and
the detecting portion is configured to detect force applied to the fixing member from the fixing gear.

7. The electrically bending endoscope according to claim 6,
wherein the fixing gear and the fixing member are formed integrally with each other.

8. The electrically bending endoscope according to claim 6,
wherein the fixing gear includes a fixing gear teeth portion provided in the fixing gear and is configured to be changed over between the fixing state and a releasing state where the fixing gear is rotatable about the central axis of the fixing gear, and
the fixing member includes a fixing member teeth portion provided in the fixing member and is configured to be changed over between a fixing state where the fixing member teeth portion is engaged with the fixing gear teeth portion and the fixing member keeps the fixing gear in the fixing state and a releasing state where the fixing member teeth portion is disengaged with the fixing gear teeth portion and the fixing member makes the fixing gear in the releasing state.

9. The electrically bending endoscope according to claim 7,
wherein the detecting portion is configured to detect force in a rotational direction of the fixing gear applied to the fixing member.

10. The electrically bending endoscope according to claim 8,
wherein the detecting portion includes a fulcrum portion supporting the fixing member rotatably about a rotational axis, the fixing member includes an application portion arranged opposite to the fixing member teeth portion relative to the rotational axis in the fixing member, and the detecting portion is configured to detect force applied to the application portion.

11. The electrically bending endoscope according to claim 8,
wherein the detecting portion is configured to detect force applied to the fixing member in a direction substantially orthogonal to the rotational direction of the fixing gear.

12. The electrically bending endoscope according to claim 1,
wherein the detecting portion is provided in the driving coupling portion and configured to detect force applied to the driving coupling portion.

13. The electrically bending endoscope according to claim 1,
wherein the driving unit includes: a driving mechanism formed by the driving portion and the driving transmission mechanism; and a supporting portion supporting the driving mechanism, and
the detecting portion is provided between the supporting portion and the driving mechanism and configured to detect reaction force applied to the driving mechanism from the driven transmission mechanism.
